(19) United States
(12) Patent Application Publication
(10) Pub. No.: US 2007/0037213 A1
(43) Pub. Date: Feb. 15, 2007

(54) Method of analyzing protein and kit for analyzing protein

(76) Inventors: Yukie Sasakura, Hitachinaka (JP); Katsuhiro Kanda, Hitachinaka (JP)

Correspondence Address: MCDERMOTT WILL & EMERY LLP, 600 13TH STREET, N.W., WASHINGTON, DC, 20005-3096, US

(21) Appl. No.: 11/498,279

(22) Filed: Aug. 3, 2006

(30) Foreign Application Priority Data
Aug. 3, 2005 (JP) 2005- 225260

(51) Int. Cl. G01N 33/53

(52) U.S. Cl. 435/7.1

(57) ABSTRACT

The expression and the phosphorylation level of a target protein are determined without performing complicated operations involving risk; the expression and the phosphorylation level of the target protein are simultaneously determined, by separating and detecting the specific protein from other proteins different from the specific protein, and by specifically detecting a phosphorylated residue of the identified target protein; and finally a ratio of the target protein phosphorylated is calculated to obtain the phosphorylation ratio of the protein.

### BACKGROUND OF THE INVENTION

1. Field of the Invention

The present invention relates to a method of analyzing a protein, and more particularly to a method of analyzing the phosphorylation state of a protein.

2. Description of the Related Art

With an enormous amount of genomic information being accumulated, the focus of life science has been shifting toward proteomics from genomics. Proteins are synthesized in cells by transcription and translation from DNA. Thereafter, through processes such as splicing, folding, and post-translation modification, the proteins change into matured proteins, and thus can fulfill the functions thereof. Post-translation modification includes various types of processes such as phosphorylation, methylation and glycosylation, and modification occurring most frequently is phosphorylation modification. It is supposed that a third of proteins of a eukaryote are phosphorylated in some way (Trends Biotechnol. 2002 Jun.; 20(6): 261-8). Phosphorylation is reversible in many cases. Proteins are activated by phosphorylation and thus fulfill functions thereof, and are inactivated by dephosphorylation. An enzyme which phosphorylates a protein is referred to as kinase, and an enzyme which dephosphorylates a protein is referred to as phosphatase. It is known that a human being has not less than five hundred types of kinases and not less than one hundred types of phosphatases. Depending on the kinases and the phosphatases, the phosphorylation state in an organism is stringently controlled, and thus the normal mechanism within the organism can be maintained. On the other hand, it has been reported that various diseases such as cancer are caused when abnormal control on phosphorylation occurs. For this reason, in drug development studies, therapeutic agents with which phosphorylation can be controlled have been searched. In clinical fields, it is expected that the phosphorylation state of a target protein will be used as markers for diagnosis of diseases.

Conventionally, a procedure described below has been practiced to determine the phosphorylation state. A radioactive molecule i³²P is incorporated into cells, and then the cells are cultivated. Thereafter, molecules of extracted proteins are separated from one another by two-dimensional electrophoresis, and are detected by use of Coomassie dye or the like. Furthermore, the existence of ³²P incorporated in the proteins is determined by autoradiography. As another method of detecting phosphorylated proteins, Western blotting can be also used in which a protein sample is transferred to a membrane after electrophoresis to detect proteins by means of antibodies recognizing phosphorylation sites.

In addition, as a technique for determining the phosphorylation state using a protein microarray, such a procedure as disclosed in Japanese Patent Application Laid-open No. Tokkai 2005-69788 has been reported. In the procedure, an antibody specific to a target protein sample is previously immobilized on a support, and then the sample is added to the support so that the target protein is captured on the support by use of the antigen-antibody reaction. Thereafter, phosphorylation is determined by use of an antibody recognizing phosphorylation sites.

It has been empirically found that the expression level of each disease marker of protein varies depending on diseases. However, each of the disease markers is not always directly involved in a mechanism for causing a disease. On the other hand, abnormality in phosphorylation intimately relates to causes of diseases. Thus, regulation for normal phosphorylation state leads to treatments for diseases. In some cases, causes of a single disease are different depending on patients. Accordingly, by determining the phosphorylation state of protein of a patient to determine a cause of a disease before starting medical treatments, it is possible to prospect effects of therapeutic agents to determine a treatment policy, and it is possible to observe the prognosis.

For the purpose of developing therapeutic agents and methods of medical treatments as described above, it is important to determine the phosphorylation state of a target protein, and it is required to develop techniques realizing accurate determination with high throughput. With respect to determination of the phosphorylation state of protein, it is important to determine the phosphorylation level of protein. In addition, it is also important to determine a proportion of a phosphorylated target protein to the expression level of the target protein in order to analyze phosphorylation and dephosphorylation as control mechanisms of organisms.

However, the conventional method using ³²P has complicated experimental processes. In addition, special facilities are required for treating ³²P which is a radioactive molecule. Thus risk is involved in treating ³²P. Because of the risk, it is not possible to administer ³²P to a human body. Thus, targets of interest are limited to cultivated cells and laboratory animals. In the case of Western blotting, it is not possible to simultaneously determine the expression level of a target protein, and hence it is required to perform an additional quantitative experiment of protein. In addition, with the method described in Japanese Patent Application Laid-open No. 2005-69788, it is possible to determine the phosphorylation level of a target protein while it is not possible to determine the expression level of the target protein. Thus, it is impossible to know the ratio of the phosphorylated target protein (phosphorylation ratio). Since the expression level of target protein varies depending on individuals, it is only possible to determine whether phosphorylation is normal or abnormal by determining not the phosphorylation level but the phosphorylation ratio. Accordingly, in order to use phosphorylation analysis for clinical diagnosis and drug development, it is essential to determine the phosphorylation level as well as the expression level of a target protein.

Patent Document: Japanese Patent Laying-open 2005-69788

Non Patent Document: Trends Biotechnol, 2002 Jun.; 20(6): 261-8

### SUMMARY OF THE INVENTION

An object of the present invention is to provide a method of determining the expression level of a target protein and the phosphorylation level of a target protein without performing complicated operations involving risk, and an analysis kit used for the method.

In the present invention, a target protein to be analyzed is identified from proteins which are different from the target protein, and then phosphorylated residue in the identified target protein is specifically detected. Accordingly, the expression level and the phosphorylation level of the target protein are simultaneously determined. Thereafter, based on the expression level and the phosphorylation level of the target protein, the ratio of the phosphorylated target protein is calculated to determine the phosphorylation ratio of the target protein.

With the present invention, the expression level and the phosphorylation level of a protein are simultaneously determined, the levels conventionally being determined separately. Accordingly, it is possible to simplify experimental operations, and thus to increase the throughput of determination. In addition, it is possible to halve the amount of samples used for the experiment. The present invention is effectively used in various fields such as examination, diagnosis, drug development, and basic studies.

### BRIEF DESCRIPTION OF THE DRAWINGS

FIG. 1 is a flow chart of determination of the phosphorylation state of a target protein.

FIG. 2 is a schematic view illustrating determination principles of simultaneously detecting the expression level and the phosphorylation level of the target protein.

FIG. 3 is a schematic view of operations for experiment using microarray.

FIG. 4A to 4D are views showing the results of an experimental example.

FIG. 5 is a flow chart of clinical diagnosis based on the phosphorylation state of a protein.

FIG. 6 is a schematic view of clinical diagnosis based on the phosphorylation state of the protein.

FIG. 7 is a flow chart of development of therapeutic agents aimed at the protein phosphorylation.

FIG. 8 is a schematic view of the development of therapeutic agents aimed at protein phosphorylation.

FIG. 9 is a schematic view of medical treatments based on the protein phosphorylation.

FIG. 10 is a schematic view of searching for phosphoresced proteins which cause diseases.

FIGS. 11A and 11B are schematic views of kits for determining a phosphorylation state.

### DETAILED DESCRIPTION OF THE PREFERRED EMBODIMENTS

A method for separating proteins from one another needs to be a method in which it is possible to purify proteins without losing a minute level of a target protein in a sample. There are methods such as, for example, electrophoresis, liquid chromatography, immunoprecipitation, and molecular array. Molecular array is considered to be one of the embodiments in which the present invention becomes most effective. A method with the following procedure can be considered. A molecule specifically bound to a target protein of interest is immobilized on a support, and then the surface of the support is blocked as required. Thereafter, a sample such as tissues extracted is added to the support so that the target protein in the sample is captured on the support. Subsequently, a molecule specifically recognizing a phosphorylation site of protein is added to the support in order to detect the phosphorylation site of the target protein.

In the case of using a molecular array described above, any solid material can be used as a support as long as a capture molecule can be immobilized. For example, it is possible to use a membrane such as nitrocellulose or PVDF, glass, silicon such as a wafer, resin such as plastic, and metal, all of which are modified appropriately, if required, for immobilizing a capture molecule. For instance, poly-L-Lysine or aminosilane with which a target molecule can be immobilized by physical adsorption, a functional group such as an aldehyde group or an epoxide group capable of immobilizing a target molecule by covalent binding, or avidin or Ni-NTA capable of immobilizing a target molecule by use of affinity to the target molecule can be used for the modification. In addition, a solid material formed of thin layers each having a hydrophilic porous matrix, such as polyacrylamide gel or agarose gel, can be used.

As a capture molecule to be immobilized on the support, a molecule capable of capturing a target molecule on the support by specifically being bound to the target molecule is used. Note that the capture molecules have to be bound to the target protein not depending on the phosphorylation state of the target molecule. As the capture molecules, for example, there can be listed an antibody, an antibody-like molecule, a protein, a peptide, nucleic acid, a nucleic-acid-like molecule, or aptamer.

Depending on properties of the support, the sample and a reagent, a blocking agent is used for preventing an increase of non-specific signals. The non-specific signals are caused because molecules other than the target molecules are adsorbed on a portion of the support on which the target molecule is not immobilized. Generally, an inactivated protein such as Bovine Serum Albumin (BSA) or a polymer such as Polyethylene Glycol (PEG) or Polyvinyl Alcohol (PVA) is used. However, for instance, although a protein of skimmed milk having a phosphate group is generally used as the blocking agent, the protein is not suitable for the present invention.

The detecting method needs to be a method with which the expression level and the phosphorylation level of a target protein can be simultaneously determined from a single sample. In the case of using a molecular array as described above, for instance, the phosphorylation ratio of a target protein can be obtained in the following manner. A sample including the target protein is previously labeled with a fluorescent dye A. An antibody which recognizes a phosphorylation site, and which is labeled with a fluorescent dye B, is used as a molecule specifically recognizing the phosphorylation site. Accordingly, the expression level of the target protein can be determined from the fluorescent dye A, and the phosphorylation level of the target protein can be determined from the fluorescent dye B, so that the phosphorylation ratio of the target protein can be obtained.

The target protein may be detected after the completion of separating the target protein of the sample, instead of previously labeling the sample. In addition, it is also possible to determine the expression level of the target protein using a molecule recognizing a site different from that recognized by the immobilized capture molecule.

In place of the labeled antibody recognizing a phosphorylation site, the combination of an unlabelled antibody recognizing a phosphorylated site and a labeled antibody specifically bound to the antibody recognizing a phosphorylation site can be used to determine the phosphorylation level. Alternatively, a dye such as ProQ Diamond (by Molecular Probe Inc.) specifically bound to a phosphate group can also be used in place of an antibody.

In addition, it may also be considered that molecular weight change resulting from the binding between an antigen and an antibody is detected by Surface Plasmon Resonance (SPR) without using labeling. SPR is resonance excitation of surface plasmon waves which mean an excited state on the surface of metal. SPR can be used to detect the molecular weight change resulting from the binding and dissociating of two molecules. SPR is advantageous in that labeling and dying are not necessary.

By referring to the drawings, descriptions will be provided for embodiments of the present invention. Note that the present invention is not limited to the embodiments.

### First Embodiment

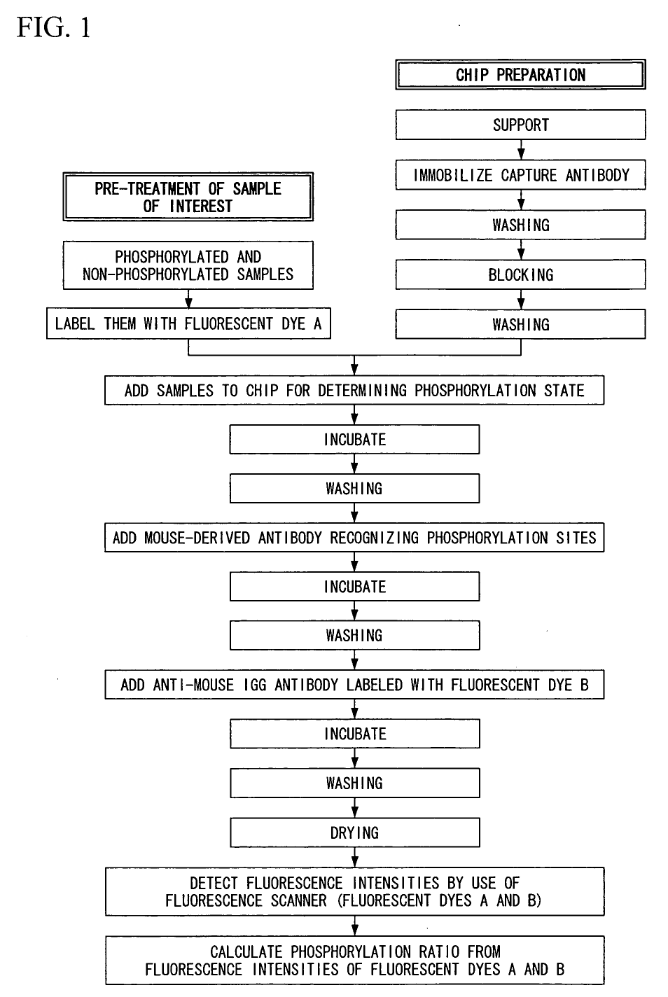

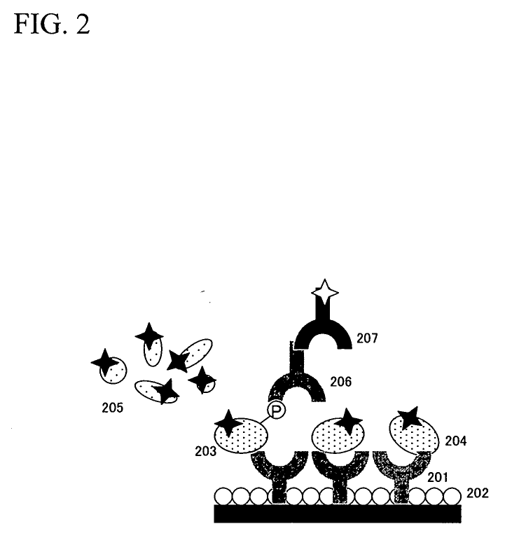

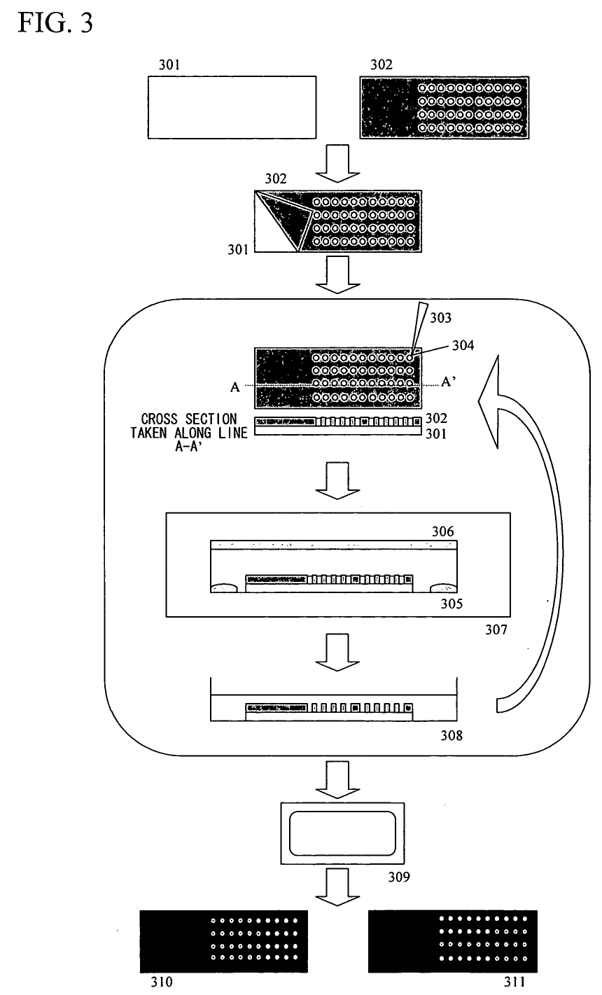

By referring to FIGS. 1 and 2, descriptions will be provided for a procedure of analyzing a phosphorylated protein using a protein microarray. FIG. 3 is a schematic diagram illustrating the experimental operations of the embodiment.

In this example, the operations are carried out in the following sequence. (1) An Antibody recognizing a target protein is immobilized on a support. (2) A sample and a control sample are added to the support. The sample contains a target protein labeled with a fluorescent dye A, and the control sample has a concentration and a phosphorylation ratio both of which are already known. (3) An antibody recognizing a phosphorylation site is added to the support. (4) An antibody which is labeled with a fluorescent dye B, and which is bound to the antibody recognizing a phosphorylation site, is added to the support. (5) The fluorescent dyes are detected. (6) The phosphorylation ratio is calculated.

In this embodiment, in addition to the above-described materials, the following are used: a spot film 302 for applying a sample or a reagent to spots on the support, a pipetter 303, an airtight container 306, a gas-phase incubator 307, a washing container 308 and a fluorescent light detection device 309.

The spot film 302 has a size of 24 mm×55 mm, and has spot holes each having a diameter of 2 mm. The holes are placed in four rows with intervals of 5 mm and in twelve columns with intervals of 4.5 mm. Since the spot film 302 is previously attached to the support, it is possible to apply a sample and a reagent in the same size to the same spots on the support. The pipetter 303 is used for applying the sample and the reagent 304 in arbitrary aliquots to the support. Since the support is placed inside the airtight container 306 together with a small amount of water drops 305, the airtight container 306 keeps moisture inside for a longer period of time to prevent the sample and the reagent on the support from evaporating. The gas incubator 307 keeps the temperature suitable for the binding reaction of the sample and the reagent. The washing container 308 is used for removing molecules not bound to the support by swaying the support together with a washing solution.

First, an antibody specific to the target protein is immobilized on the support. The spot film 302 is attached to the support 301, and an antibody 201 specific to the target protein is applied to the support by use of the pipetter 303. The antibody used in this case is bound to the target protein not depending on the phosphorylation state of the target protein. The support is placed inside the airtight container 306 together with a water drop 305, and then is placed inside the incubator 307 so that the antibody is bound to the support. Antibodies not bound to the support are removed by use of a washing operation, and then unreacted portions on the surface of the support are blocked by use of a protein 202 having no influence on the antigen-antibody reaction. Thereafter, the support is cleaned again, and thus a chip for determining the phosphorylation state is obtained.

Subsequently, a sample and a control sample are added to the support. The sample contains the target protein labeled with a fluorescent dye A, and the control sample has the concentration and the phosphorylation ratio both of which are already known. The sample of interest, which is previously labeled with a fluorescent dye A, and the control sample are applied respectively to spots different to each other on the support on which the antibody is immobilized. The support is placed inside the airtight container 306 together with water drops 305, and then is incubated inside the incubator 307 so that each of the samples is bound to the immobilized antibody. Each of phosphorylated target protein 203 and non-phosphorylated target protein 204 is captured on the support by the immobilized antibody 201. Molecules 205 other than the target protein are not captured on the support, and thus are removed by the washing operation.

Subsequently, an antibody 206 recognizing a phosphorylation site is added to the support. An antibody to phosphorylated amino acid residues (mainly a serine, a tyrosine or a threonine) in the protein is applied to the support. In this embodiment, the antibody is a mouse-derived antibody recognizing a phosphorylation site. Thereafter, the support is placed inside the airtight container 306 together with water drops 305, and then is incubated in the incubator 307 so that the antibody 206 is bound to a phosphate group in the target protein. Then, unreacted antibodies are removed by the washing operation.

Subsequently, an antibody which is labeled with a fluorescent dye B, and which is specific to the antibody recognizing a phosphorylation site, is added to the support. Specifically, an antibody 207 specific to the antibody 206 recognizing a phosphorylation site is applied to the support. The antibody 207 is previously labeled with the fluorescent dye B having excitation and emission wavelengths different from those of the fluorescent dye A. In this embodiment, the antibody 207 is an anti-mouse IgG antibody labeled with the fluorescent dye B. Thereafter, the support is placed inside the airtight container 306 together with water drops 305, and then is placed in the incubator 307 so that the antibody 207 is bound to the antibody 206 recognizing a phosphorylation site. After unreacted antibodies are removed by the washing operation, the support is dried by spraying nitrogen gas thereto.

Subsequently, fluorescence detection is carried out. The fluorescence intensities of the fluorescent dyes A and B are measured respectively by use of the excitation and emission wavelengths of each of the dyes. The expression level of the target protein and the phosphorylation level of the target protein are determined respectively from the fluorescence intensities of the fluorescent dyes A and B.

Lastly, based on the fluorescence intensity of the control sample, the level of the target protein, the phosphorylation level and the phosphorylation ratio of the sample of interest are calculated. Calibration curves of the protein concentration and the phosphorylation level of the control sample are obtained. Based on the calibration curves, the protein concentration and the phosphorylation level of the sample of interest are calculated, and thus the phosphorylation ratio is obtained by comparing the protein concentration with the phosphorylation level at last.

### EXPERIMENTAL EXAMPLE

In this experimental example, detailed descriptions will be provided for the present invention by taking an experiment as an example. In the experiment, the level of the target protein and the phosphorylation level of an enzyme, ERK2 (extracellular signal regulated kinase), are simultaneously determined. ERK2 is one of MAP kinases (mitogen-activated protein kinase), and the activation of ERK2 is regulated by phosphorylation.

Immobilization of Capture Antibody on Substrate

In the experiment, PtoteoChip (TypeA, by Proteogen Inc.) was used as a support. A spot film (Japanese Design registration No. 1213441) was previously attached to the surface of the ProteoChip. The spot film was used for applying a sample and a reagent in the same size and in the same spots on a support. A rabbit-derived anti-human ERK2 polyclonal antibody (71-1800, by Zymed Laboratories Inc.) was used as a capture antibody. The antibody was diluted with PBS (pH 7.4) containing 30% of Glycerol so that the concentration of the antibody was 100 μg/ml, and the solution was applied in 1.5 μl aliquots to the support by use of a pipetter 303.

The support was placed inside an airtight container 306 having water drops 305 at corners thereof, and was incubated overnight at 37° C. so that the capture antibody was immobilized on the support. After the immobilization, the support was immersed into 50 ml of a washing solution (PBS containing 0.5% of Tween 20, pH 7.8), and then was swayed for 10 minutes to remove antibodies not bound to the support. After the washing, the last traces of water were removed by use of filter paper.

Blocking

The support was immersed into 50 ml of PBS (pH 7.8) containing 0.5% of BSA, and then was gently swayed for one hour. After the solution was discarded, 50 ml of a washing solution was added to the airtight container 306, and the support was swayed for ten minutes to be cleaned. After the washing, the last traces of water were removed by use of filter paper.

Labeling of Sample Solution Containing Antigen

Each of phosphorylated ERK2 (14-439, by Upstate) and non-phosphorylated ERK2 (14-515, by Upstate) was diluted with PBS containing 1 mg/ml of BSA so that the concentration of the ERK2 was of 10 μg/ml, and then was labeled with a fluorescent dye, Cy3Dye labeling reagent-NHS(Q13008, by Amersham Bioscience). By gel filtration using SephadexG25 (17-0032-01, Amersham Bioscience), each of ERK2s is separated from free dye. Thereafter, the solution is concentrated by centrifugation so that the concentration of the ERK2 was 20 μg/ml. The protein concentration and the labeling efficiency of Cy3 were obtained by measuring absorption spectrum of wavelength of 250 nm to 700 nm to obtain the respective absorption maximum wavelengths of 280 nm and 556 nm.

Capturing of Antigen by Capture Antibody

By use of a pipetter 303, each of the solution of the labeled phosphorylated ERK2 and the solution of the labeled non-phosphorylated ERK2 was applied in 1.5 μl aliquots to the support on which the capture antibody was immobilized. The support was placed inside the airtight container 306 having water drops 305 at corners thereof, and was incubated for 1.5 hour at 37° C. so that the antigen and the capture antibody reacted with each other. After the antigen solution was removed by aspiration, the support was immersed into 50 ml of a washing solution, and was swayed for 15 minutes to remove unreacted antigens. Thereafter, the last traces of water were removed by use of filter paper.

Capturing of Phosphorylated Protein by Antibody Recognizing Phosphorylation Site

Each of a solution containing a mouse-derived anti-phosphothreonine antibody (13-9200, by Zymed Laboratories Inc.) and a solution containing an anti-phosphotyrosine antibody (9200, by Zymed Laboratories Inc.) was prepared by use of PBS (pH 7.4) containing 30% of Glycerol and 10% of BSA, the concentrations of each of the antibodies each being of 100 μg/ml. By use of the pipetter 303, each of the solutions was applied in 1.5 μl aliquots to the support. The support was placed inside the airtight container 306 having water drops 305 at corners thereof, and was incubated for 1.5 hour at 37° C. so that the antibody reacted with the antigen. After the antibody solution was removed by aspiration, the support was immersed into 50 ml of a washing solution, and was swayed for 15 minutes to remove unreacted antibodies. Thereafter, the last traces of water were removed by use of filter paper.

Reaction with Labeled Antibody

A solution of a goat-derived anti-mouse IgG polyclonal antibody labeled with Cy5(81-6516, by Zymed Laboratories Inc.) was prepared by use of PBS (pH 7.4) containing 30% of Glycerol and 10% of BSA so that the concentration of the antibody was of 2 μg/ml. The solution was applied in 1.51 μl aliquots to the support by means of the pipetter 303. The support was placed inside the airtight container 306 having water drops 305 at corners thereof, and was incubated for 30 minutes at 37° C. so that the antibodies reacted with the antibodies recognizing phosphorylation site. After the antibody solution was removed by aspiration, the support was immersed into 50 ml of a washing solution, and was swayed for 15 minutes to remove unreacted antibodies. After the washing, the last traces of water were removed by spraying a nitrogen gas to the support, and then the spot film was removed.

Detection by Use of Fluorescence Scanner

The support was scanned by use of a scanner for fluorescence detection, ScanArray Express (by Packard BioScience, Co.). The image was analyzed by means of QuantumArray (by Packard BioScience, Co.). The fluorescence intensity of each spot was converted into numeric values. The total amount of ERK2 in the sample solution was determined from Cy3 (an excitation wavelength of 550 nm, a emission wavelength of 570 nm), and the phosphorylation level of ERK2 was determined from Cy5 (an excitation wavelength of 650 nm, a emission wavelength of 680 nm).

Results of Experiment

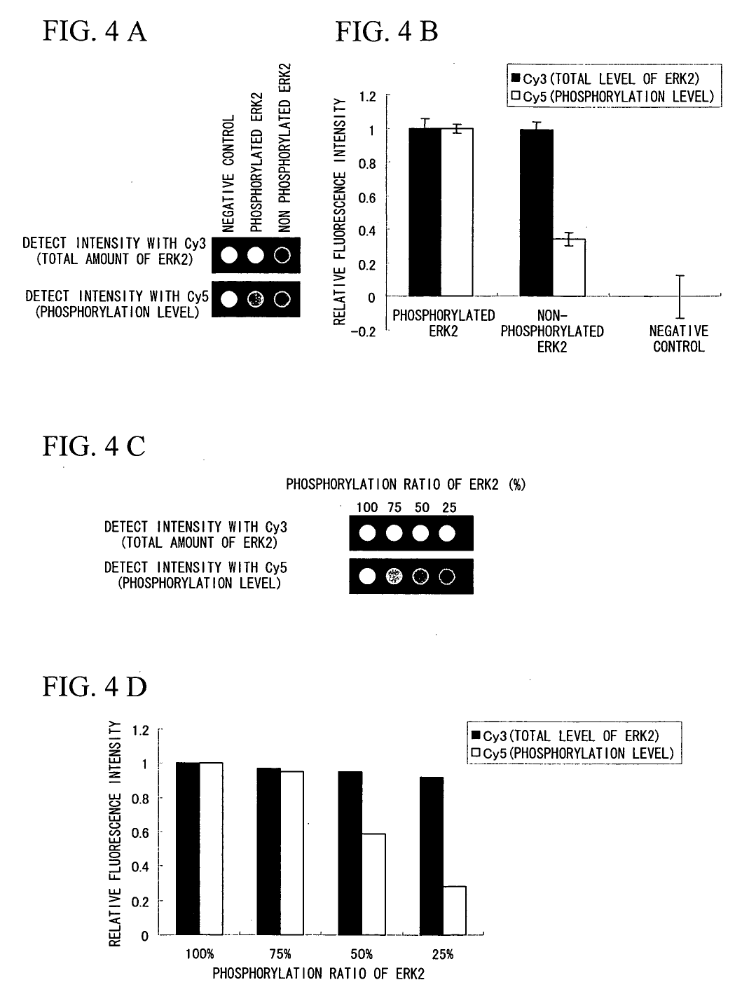

A schematic view of an image of fluorescence scanning using Cy3 is shown in the upper view of FIG. 4A. A schematic view of an image of fluorescence scanning using Cy5 is shown in the lower view of FIG. 4A. The scanning images were converted into numeric values each indicating fluorescence intensity by use of analysis software. The value of fluorescence intensity of negative control was subtracted as background from each of values of the fluorescence intensities of the samples. Thereafter, the obtained values were indicated as relative values with the value of fluorescence intensity of the phosphorylated ERK2 defined as 1 (FIG. 4B). When the non-phosphorylated ERK2 was examined, the fluorescence intensity thereof detected with Cy3 was almost the same as that of the phosphorylated ERK2. Thus, it was acknowledged that the total level of the non-phosphorylated ERK2 and the total level of the non-phosphorylated ERK2 are the same. Concurrently, from the fluorescence intensity determined with Cy3, it was acknowledged that the phosphorylation level of the non-phosphorylated ERK2 was significantly reduced to 34%. In addition, based on the results, the same experiment was carried out using ERK2s prepared to have phosphorylation ratios respectively of 100%, 75%, 50% and 25% as shown in FIG. 4C. It was acknowledged that although the total level of ERK2 was almost the same for each of the types, each of the phosphorylation levels increased and decreased depending on the concentration (FIG. 4D). Because of the results, it was acknowledged that this method can be used for accurately determining the expression level and the phosphorylation level of a protein.

### Second Embodiment

Kit for Determining Phosphorylation State

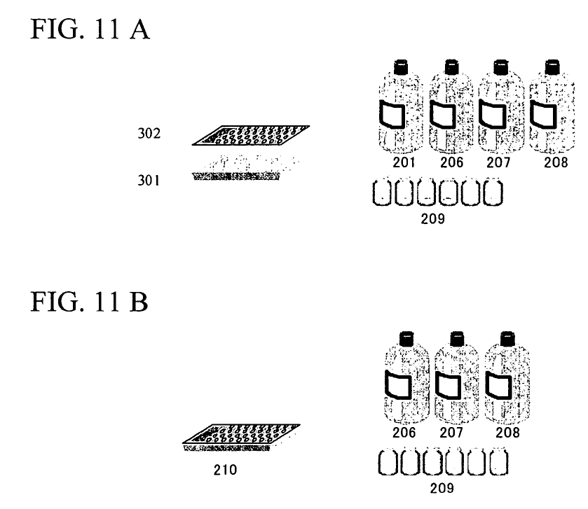

FIGS. 11A and 11B are explanatory views showing examples of kit for analyzing protein, the kit being used for easily carrying out the protein analysis of the present invention.

The determination kit shown in FIG. 11A is used for determining the phosphorylation state, and includes a support 301, a spot film 302; a capture antibody 201 to a target protein; a reagent 208 for labeling a sample of interest with a fluorescent dye A; a control sample 209 previously labeled with a fluorescent dye A; an antibody 206 recognizing a phosphorylation site; and an antibody 207 which is labeled with the fluorescent dye B, and which specifically recognizes the antibody 206.

A user of the determination kit first attaches the spot film 302 to the support 301 to immobilize the capture antibody 201 to a target protein on the support. The user labels a sample of interest with the reagent 208 for labeling the sample with the fluorescent dye A, the reagent 208 being included in the kit. The user adds the sample of interest and the control sample 209 included in the kit to the support in order to immobilize a target protein on the support. The user sequentially adds, to the support, the antibody 206 recognizing a phosphorylation site and the antibody 207, which is labeled with the fluorescent dye B, and which specifically recognizes the antibody 206. Lastly, the user detects fluorescence intensity of the sample. Based on the fluorescence intensities of the control sample, calibration curves are created to obtain the level of the target protein and the phosphorylation level of the target protein in the sample of interest. Thereafter, the phosphorylation ratio is calculated.

The determination kit illustrated in FIG. 11B includes a support 210 having a capture antibody to a target protein immobilized on the support 210; a reagent 208 for labeling a sample of interest with a fluorescent dye A; a control sample 209 previously labeled with a fluorescent dye A; an antibody 206 recognizing a phosphorylation site; and an antibody 207 which is labeled with the fluorescent dye B, and which specifically recognizes the antibody 206.

A user of the determination kit labels a sample of interest with the reagent 208 for labeling the sample with the fluorescent dye A, the reagent 208 being included in the kit. The user adds the sample of interest and the control sample 209 included in the kit to the support in order to immobilize the target protein on the support. The user sequentially adds, to the support, the antibody 206 recognizing a phosphorylation site, and the antibody 207 which is labeled with the fluorescent dye B, and which specifically recognizes the antibody 206. Lastly, the user detects the fluorescence intensity of the sample. Based on the fluorescence intensities of the control sample, calibration curves are created to obtain the level of the target protein and the phosphorylation level of the target protein in the sample of interest. Thereafter, the phosphorylation ratio is calculated.

### Third Embodiment

Determination of Phosphorylation State by Use of Electrophoresis

Molecules of a sample containing a target protein previously labeled with a fluorescent dye A are separated from one another in gel by electrophoresis based on molecular sizes, the isoelectric point, or the like. Subsequently, the proteins in the gel are transferred to a nitrocellulose membrane or the like, and then portions of the membrane on each of which no protein is bound are blocked. Thereafter, the membrane is immersed into a solution containing an antibody 206 recognizing a phosphorylation site so that the antibody 206 is bound to a phosphate group in the protein. The membrane is then immersed into a solution containing an antibody 207 which is labeled with a fluorescent dye B, and which is specific to the antibody recognizing a phosphorylation site, so that the antibody 207 is bound to the antibody recognizing a phosphorylation site. The fluorescence intensities of the respective fluorescent dyes A and B are measured by use of the excitation and emission wavelengths of each of the fluorescent dyes. Based on the molecular weight and the isoelectric point of the target protein, the location of the target protein on the support is determined. The expression level of the target protein is determined from the protein band or the fluorescence intensity of the fluorescent dye A. The phosphorylation level of the target protein is determined from the fluorescence intensity of the fluorescent dye B. Thus, the phosphorylation ratio is calculated.

### Fourth Embodiment

Clinical Diagnosis Based on Phosphorylation

In this embodiment, descriptions will be provided for a method of giving a diagnosis of the presence or absence of diseases and the types or stages of the diseases. The diagnosis is given by determining the phosphorylation state of a specific protein in a sample obtained from a patient, by means of the kit for determining the phosphorylation state of the present invention.

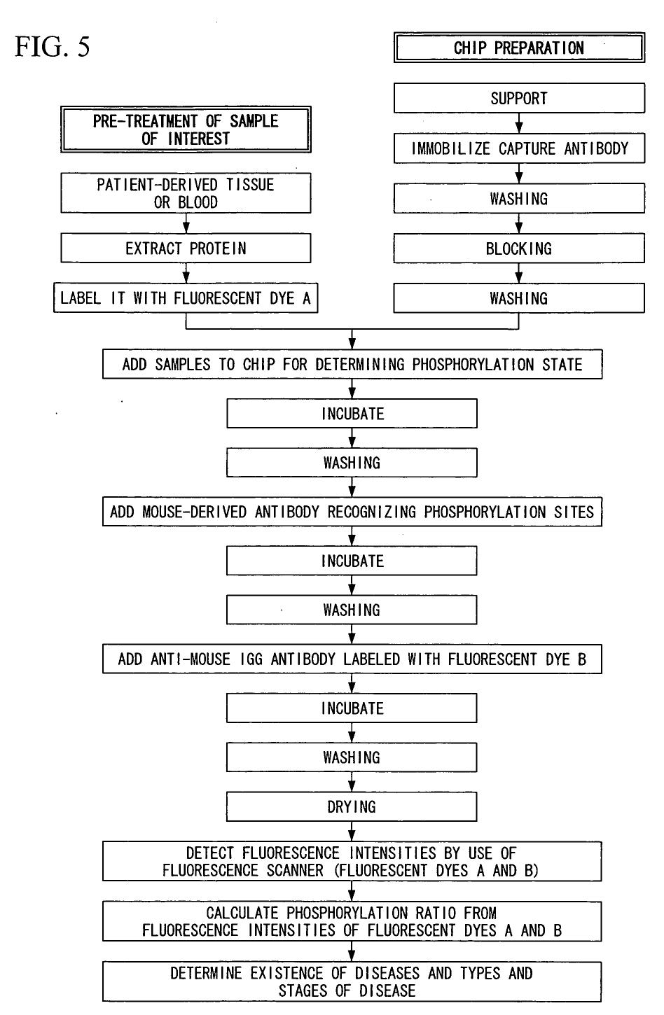

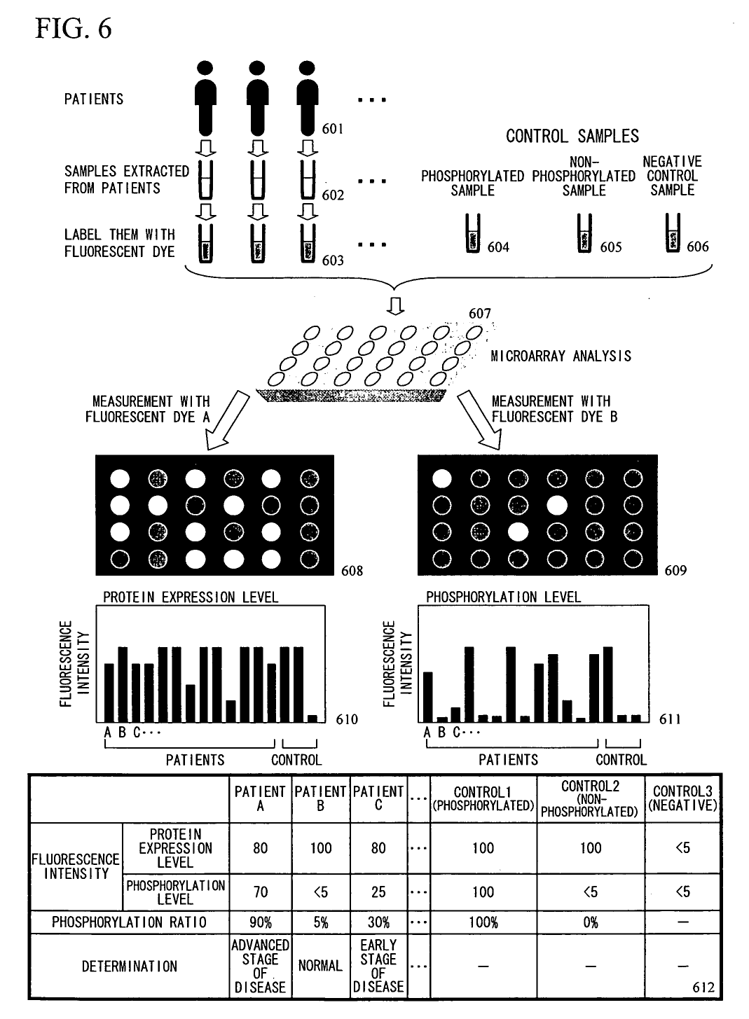

FIG. 5 shows the flow of the determination in the embodiment. FIG. 6 shows a schematic view of the determination and an example of data to be displayed.

Extraction of Protein From Tissue

Cells of interest are cut respectively from tissues isolated respectively from individual patients 601 by operation or biopsy. The cells are added to homogenizing buffer (for instance, 0.25M saccharose), and then are homogenized by a homogenizer. Sediments are removed by centrifugation, and thus samples 206 are obtained. Alternatively, appropriate process is applied to blood sampled to use the blood as a sample in some cases. The samples 602 are labeled respectively with a fluorescent dye A to obtain samples 603 each labeled with the fluorescent dye A.

Determination of Phosphorylation State

The mobilization of a capture antibody and the determination of the phosphorylation state of each of the samples are carried out by use of the kit for determining the phosphorylation state. In the case where the kit for determining the phosphorylation state shown in FIG. 1B is used, for instance, any of the samples of interest which are prepared as described above, a phosphorylated control sample 604, a non-phosphorylated control sample 605, and a negative control 606, which are included in the kit, are added to the support. Accordingly, a target protein is captured on the support. Subsequently, an antibody 206 recognizing a phosphorylation site and an antibody 207 which is labeled with a fluorescent dye B, and which specifically recognizes the antibody 206, are sequentially added to the support.

A chip 607 for phosphorylation state determination obtained by completing the above-described process is scanned with excitation light of the respective fluorescent dyes A and B. Accordingly, images 608 and 609 respectively showing fluorescence intensity distributions of the respective fluorescent dyes A and B are obtained. The images are converted into numeric values by use of analysis software to obtain graphs 610 and 611. The graph 610 shows comparative information on the expression level of proteins of the respective samples extracted from the respective patients. The graph 611 shows comparative information on the levels of phosphorylation of the respective samples extracted from the respective patients. The phosphorylation ratios of the respective samples are calculated based on the expression level and the levels of phosphorylation of the target proteins in the respective samples extracted from the respective patients. Subsequently, determination is made on the presence or absence of diseases and the types or stages of the diseases. A table 612 shown in the lower part of FIG. 6 collectively shows the results of comparison among the phosphorylation ratios of the samples extracted respectively from the patients, and the results of the diagnosis thereof.

### Fifth Embodiment

Drug development Intended for Phosphorylation

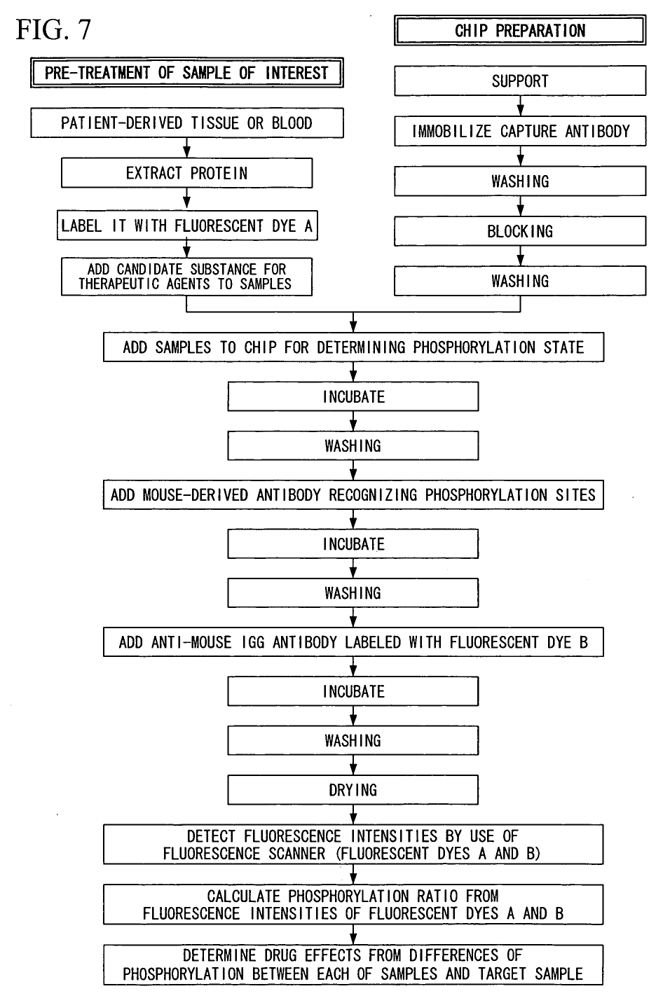

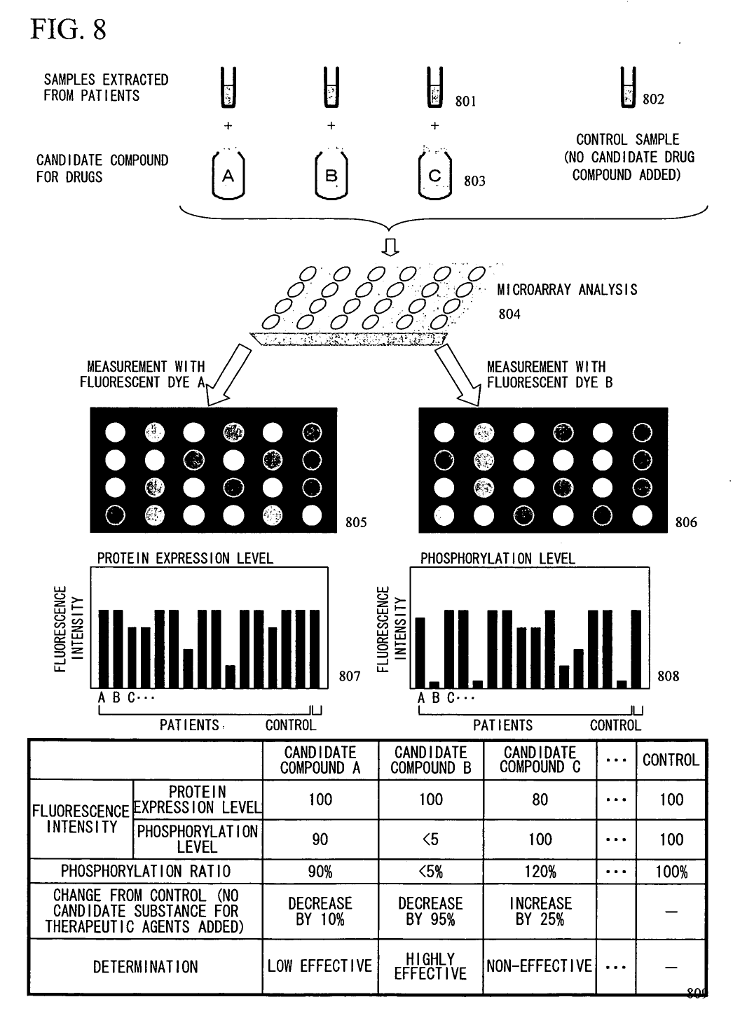

Descriptions will be provided for the case where drugs intended for phosphorylation are developed by searching for candidate molecules for therapeutic agents, with which phosphorylation can be regulated, by use of the kit for determining phosphorylation state of the present invention. FIG. 7 shows the flow of the determination in the embodiment. FIG. 8 shows a schematic view of the determination and an example of data to be displayed.

Various candidate molecules 803 for therapeutic agents are added respectively to the samples 801 extracted from a patient. Each of the sample shows an abnormal phosphorylation state. With a control sample 802 to which no molecule is added, analysis of phosphorylation state is carried out in accordance with the method of the first embodiment, by use of a chip 804 for determining the phosphorylation state. The chip is scanned with each of excitation wavelengths of respective fluorescent dyes A and B so that images 805 and 806 respectively showing fluorescence intensity distributions of the respective fluorescent dyes A and B are obtained. The images are converted into numeric values by use of analysis software to obtain graphs 807 and 808. The graph 807 shows comparative information on the expression level of the proteins of the respective samples. The graph 808 shows comparative information on the phosphorylation levels of the respective samples extracted from the respective samples. The phosphorylation ratios of the respective samples to which the respective drugs are added are calculated based on the expression level and the phosphorylation level of the target proteins in the respective samples. From the differences of the phosphorylation ratios of the control sample and the candidate molecules, effects of the respective candidate molecules for therapeutic agents are determined. A table 809 shown in the lower part of FIG. 8 collectively shows the results of comparison of the influences on the respective phosphorylation ratios among the candidate molecules, and the results of determination of drug effects thereof.

### Sixth Embodiment

Medical Treatments Depending on Phosphorylation State

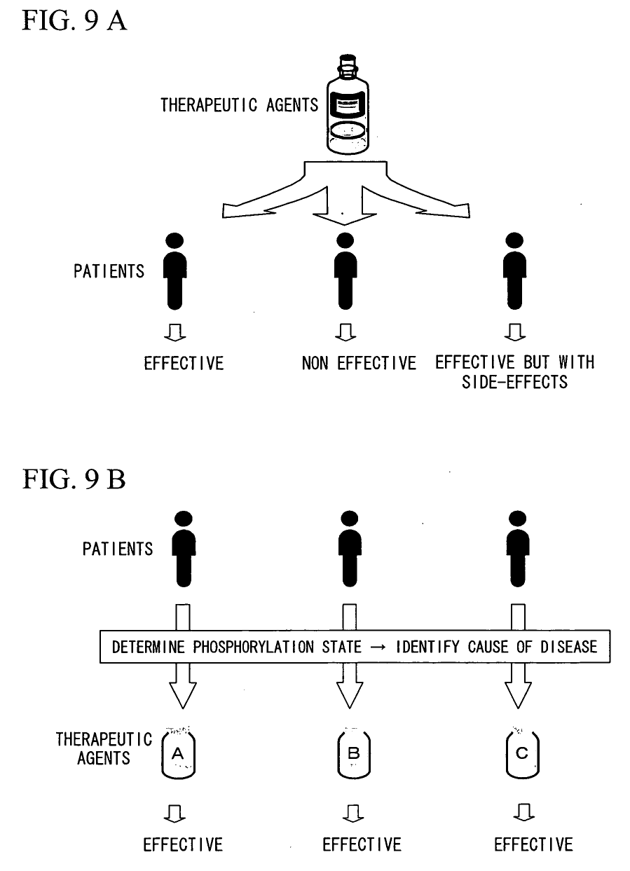

As shown in FIG. 9A, for instance, in the case where a single therapeutic agent is administered to each of patients suffering from a single disease resulting respectively from different causes, it is possible that effect of the therapeutic agent varies depending on the individual patients. It is also possible that side effects are caused which affect normal functions irrelevant to the disease depending on patients.

However, as illustrated in FIG. 9B, it is possible to select a suitable therapeutic agent for each patient by previously determining the phosphorylation state of each patient by use of the kit for determining a phosphorylation state of the present invention in order to identify a protein resulting in a disease. In addition, by using therapeutic agents acting on only the cause of the disease, it is possible to reduce side effects affecting other normal functions.

### Seventh Embodiment

Identification of Phosphorylation Resulting in Disease

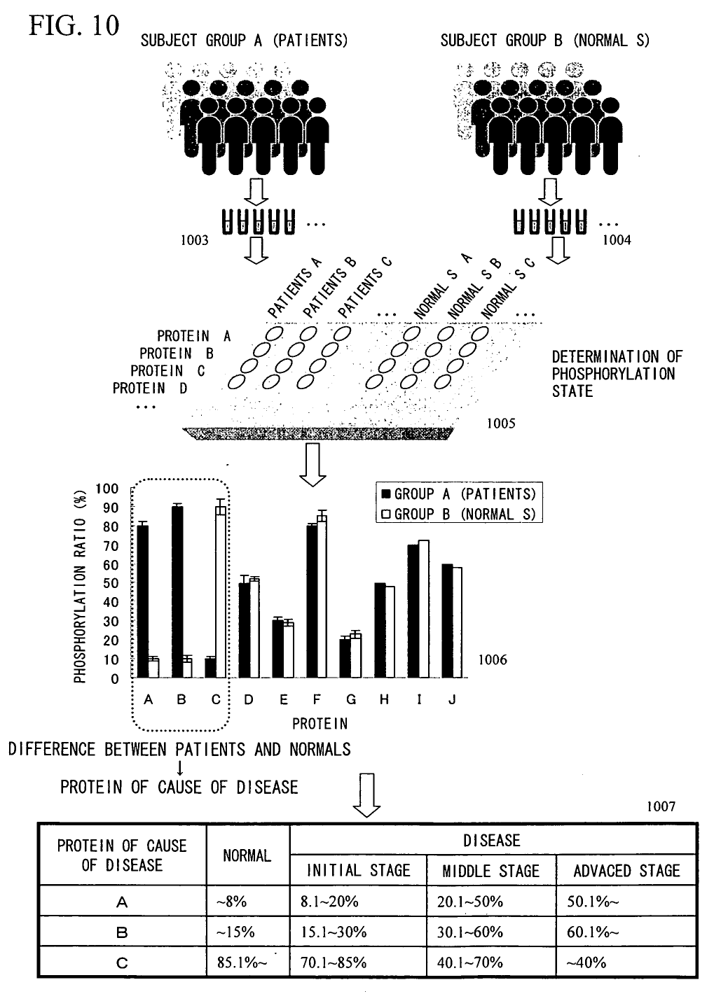

As shown in FIG. 10, for instance, the present invention can be also used to find the cause of a disease whose onset mechanism is not clear. Samples 1003 are obtained from a subject group A consisting of a predetermined number of patients, and samples 1004 are obtained from a subject group B consisting of a predetermined number of normal individuals. By determining phosphorylation states of a plurality of types of proteins of the samples 1003 and 1004 by use of the kit for determining phosphorylation state of the present invention, it is possible to regard a protein having different phosphorylation states in the group A and the group B as a candidate of a protein resulting in the disease. In addition, since the case has been reported that phosphorylation varies depending on stages of a disease, it is possible to regard phosphorylation as a diagnostic standard of stages of a disease.

What is claimed is:

1. A method of analyzing a protein comprising the steps of:
determining the amount and the phosphorylation level of a specific protein in a sample respectively by use of methods different from each other;
calculating a ratio of the protein phosphorylated from the determined the amount and the phosphorylation level of the protein; and
obtaining a phosphorylation ratio of the protein.

2. The method of analyzing a protein according to claim 1, further comprising the steps of:
detecting the specific protein by separating the specific protein from proteins deferent from the specific protein; and
specifically detecting a phosphorylation site of the specific protein.

3. The method of analyzing a protein according claim 2, further comprising the steps of:
capturing the specific protein on a support on which molecules are immobilized, the molecules specifically bound to the protein regardless of a phosphorylation state of the protein; and
specifically detecting the phosphorylation site of the protein to determine the the amount and the phosphorylation level of the protein.

4. The method of analyzing a protein according claim 3, wherein an antibody is used as the molecules specifically bound to the specific protein regardless of the phosphorylation state of the protein.

5. The method of analyzing a protein according to claim 1, wherein, for the method of determining the phosphorylation level, molecules specifically bound to a phosphate group in the protein are used.

6. The method of analyzing a protein according to claim 5, wherein an antibody recognizing a phosphorylation site is used as the molecules specifically bound to the phosphate group in the protein.

7. The method of analyzing a protein according to claim 1, wherein, for the method of determining the amount and the phosphorylation level of the specific protein separately, the amount and the phosphorylation level of the protein are determined separately by labeling a sample containing the specific protein with a first fluorescent molecule, and by labeling molecules specifically bound to a phosphate group with a second fluorescent molecule having excitation and emission wavelengths different from those of the first fluorescent molecule.

8. A kit for analyzing a protein comprising:
a support;
molecules specifically bound to a specific protein regardless of a phosphorylation state of the protein;
a reagent for labeling a sample of interest with a fluorescent dye A;
a control sample previously labeled with the fluorescent dye A; and
molecules which are labeled with a fluorescent dye B having excitation and emission wavelengths different from those of the fluorescent dye A, and which specifically recognize a phosphate group.

9. The kit for analyzing a protein according claim 8, wherein an antibody is used as the molecules specifically bound to the specific protein regardless of the phosphorylation state of the protein.

10. The kit for analyzing a protein according to claim 8, wherein an antibody recognizing a phosphorylation site is used as the molecules specifically bound to the phosphate group in the protein.

11. A kit for analyzing a protein comprising:
a support on which molecules are immobilized, the molecules specifically bound to a specific protein regardless of a phosphorylation state of the protein;
a reagent for labeling a sample of interest with a fluorescent dye A;
a control sample previously labeled with the fluorescent dye A; and
molecules which are labeled with a fluorescent dye B having excitation and detection wavelengths different from those of the fluorescent dye A, and which specifically recognize a phosphate group.

12. The kit for analyzing a protein according claim 11, wherein an antibody is used as the molecules specifically bound to the specific protein regardless of the phosphorylation state of the protein.

13. The kit for analyzing a protein according to claim 11, wherein an antibody recognizing a phosphorylation site is used as the molecules specifically bound to the phosphate group in the protein.
